SEQUENCE LISTING (4 sequences)

SEQ ID NO: 1 - protein, 247 aa, Artificial Sequence
  Antigen
MAKNKNVGKPRNYKLASGVVRFGKSKMYHKKAIYKFLKKTTPKKVEASKPAFVEKKVGGAKNGGTRMVRVKKLKNDFPTMERRAHRIAKKPEKLSRRVRPTLTPGTIAVILAGIHKGKRIVILKELSSGMLLISGPFKLNNCPIRRINQRYLLATSTKLDVSSIKMPENINDDYFRRLRAAKKPAGSVFEGKKEEYKPSEQRKKDQVEVDKQLLNVIMKHPEASLLKQYLKKSFGLSKGQYPHNMKF

SEQ ID NO: 2 - protein, 169 aa, Artificial Sequence
  Antigen
VRFGKSKMYHKKAIYKFLKKTTPKKVEASKPAFVEKKVGGAKNGGTRMVRVKKLKNDFPTMERRAHRIAKKPEKLSRRVRPTLTPGTIAVILAGIHKGKRIVILKELSSGMLLISGPFKLNNCPIRRINQRYLLATSTKLDVSSIKMPENINDDYFRRLRAAKKPAGSV

SEQ ID NO: 3 - DNA, 741 bp, Artificial Sequence
  Antigen DNA
atggcgaagaacaagaacgtcggtaagccgaggaactacaagttagcctccggagtcgtccggttcggcaaatctaaaatgtaccacaagaaggcaatctataaattcttgaagaagacaactcccaaaaaggttgaggccagtaagcccgccttcgttgagaagaaggtcggaggtgccaagaatgggggtactcgtatggttcgcgtcaagaagttgaagaacgacttccccaccatggaaagacgtgctcatagaatcgccaagaagcctgaaaagctctctcgcagggtccgtcctaccctcacccctggaactattgcagttattcttgcaggtatccacaaaggaaagagaatcgtcattctcaaggagctctccagtggaatgcttctgatttctggccccttcaagcttaataactgcccaattagaaggattaatcaacgctatttgttggccacatcaaccaagctcgatgtttcatccattaaaatgcccgagaacattaatgatgattacttccgtcgtttaagagccgccaagaagccagctggtagtgtattcgaaggtaaaaaggaagaatacaaaccttctgaacaacgtaagaaggaccaagtcgaagttgataagcagctcctcaatgtcatcatgaagcaccccgaagcctctcttttgaaacaatacttgaagaagtccttcggtcttagcaagggacaatatcctcataatatgaaattt

SEQ ID NO: 4 - DNA, 507 bp, Artificial Sequence
  Antigen DNA
gtccggttcggcaaatctaaaatgtaccacaagaaggcaatctataaattcttgaagaagacaactcccaaaaaggttgaggccagtaagcccgccttcgttgagaagaaggtcggaggtgccaagaatgggggtactcgtatggttcgcgtcaagaagttgaagaacgacttccccaccatggaaagacgtgctcatagaatcgccaagaagcctgaaaagctctctcgcagggtccgtcctaccctcacccctggaactattgcagttattcttgcaggtatccacaaaggaaagagaatcgtcattctcaaggagctctccagtggaatgcttctgatttctggccccttcaagcttaataactgcccaattagaaggattaatcaacgctatttgttggccacatcaaccaagctcgatgtttcatccattaaaatgcccgagaacattaatgatgattacttccgtcgtttaagagccgccaagaagccagctggtagtgta

(19) United States
(12) Patent Application Publication
(10) Pub. No.: US 2022/0118067 A1
(43) Pub. Date: Apr. 21, 2022

(54) SEA LICE VACCINES

(71) Applicant: BENCHMARK ANIMAL HEALTH LIMITED, Sheffield (GB)

(72) Inventor: Sarah Elizabeth BARKER, Edinburgh (GB)

(21) Appl. No.: 17/428,364

(22) PCT Filed: Feb. 4, 2019
(86) PCT No.: PCT/GB2019/050295

§ 371 Date: Aug. 4, 2021

(51) Int. Cl. A61K 39/00; A61P 33/14; C07K 14/435

(52) U.S. Cl. CPC A61K 39/0003; C07K 14/43509; A61P 33/14

(57) ABSTRACT

Proteins derived from secretory/excretory products of Lepeophtheirus salmonis, including recombinant proteins, DNA encoding the proteins, vaccines and antigens comprising the proteins or the DNA, and uses thereof for the prevention or treatment of sea lice (Lepeophtheirus salmonis or Caligus rogercresseyi) in fish, and related methods of treatment.

The present invention relates to proteins derived from secretory/excretory products of L. salmonis, including recombinant proteins, DNA encoding the proteins, vaccines and antigens comprising the proteins or the DNA, and uses thereof for the prevention or treatment of sea lice in fish, and related methods of treatment.

A number of closely related species of parasitic copepods in the family Caligidae (caligid copepods) infect and cause disease in cultured fish. Collectively, these species are referred to as sea lice. There are three major genera of sea lice: Pseudocaligus, Caligus and Lepeophtheirus. With respect to salmonid production throughout the northern hemisphere, one species, the salmon louse (Lepeophtheirus salmonis), is responsible for most disease outbreaks on farmed salmonids. This parasite is responsible for indirect and direct losses in aquaculture in excess of US $100 million annually. All developmental stages of sea lice, which are attached to the host, feed on host mucus, skin and blood. The attachment and feeding activities of sea lice result in lesions that vary in their nature and severity depending upon: the species of sea lice, their abundance, the developmental stages present and the species of the host (Johnson, S. C. et al., “Interactions between sea lice and their hosts”. In: Host-Parasite Interactions. Editors: G. Wiegertjes and G. Flik, Garland Science/Bios Science Publications, 2004, pp. 131-160). In the southern hemisphere, Caligus rogercresseyi, is the primary caligid affecting the salmon farming industry in Chile (Gonzalez, L. and Carvajal, J. Aquaculture 220: 101-117, 2003).

Caligid copepods have direct life cycles consisting of two free-living planktonic nauplius stages, one free-swimming infectious copepodid stage, four to six attached chalimus stages, one or two preadult stages, and one adult stage (Kabata, Z., Book 1: Crustacea as enemies of fishes. In: Diseases of Fishes., Editors: Snieszko, S. F. and Axelrod, H. R.; New York, T.F.H. Publications, 1970, p. 171). Each of these developmental stages is separated by a moult. Once the adult stage is reached, caligid copepods do not undergo additional moults. In the case of L. salmonis, eggs hatch into the free-swimming first nauplius stage, which is followed by a second nauplius stage, and then the infectious copepodid stage. Once the copepodid locates a suitable host fish, it continues its development through four chalimus stages, first and second preadult stages, and then a final adult stage (Schram, T. A. “Supplemental descriptions of the developmental stages of Lepeophtheirus salmonis (Kroyer, 1837) (Copepoda: Caligidae)”. In: Pathogens of Wild and Farmed Fish: Sea Lice. Editors: Boxshall, G. A. and Defaye, D., 1993, pp. 30-50). The moults are characterized by gradual changes as the animal grows and undertakes physical modifications that enable it to live as a free-roaming parasite, feeding and breeding on the surface of the fish.

Caligid copepods (sea lice) feed on the mucus, skin and blood of their hosts leading to lesions that vary in severity based on the developmental stage(s) of the copepods present, the number of copepods present, their site(s) of attachment and the species of host. In situations of severe disease, such as is seen in Atlantic salmon (Salmo salar) when infected by high numbers of L. salmonis, extensive areas of skin erosion and hemorrhaging on the head and back, and a distinct area of erosion and sub-epidermal haemorrhage in the perianal region can be seen (Grimnes, A. et al. J Fish Biol 48: 1179-1194, 1996). Sea lice can cause physiological changes in their hosts including the development of a stress response, reduced immune function, osmoregulatory failure and death if untreated.

There are several management strategies that have been used for reducing the intensity of caligid copepod (sea lice) infestations. These include: fallowing of sites prior to restocking, year class separation and selection of farm sites to avoid areas where there are high densities of wild hosts or other environmental conditions suitable for sea lice establishment (Pike, A. W. et al. Adv Parasitol 44: 233-337, 1999). Although the use of these strategies can in some cases lessen sea lice infection rates, their use individually or in combination has not been effective in eliminating infection.

A variety of chemicals and drugs have been used to control sea lice. These chemicals were designed for the control of terrestrial pests and parasites of plants and domestic animals. They include compounds such as hydrogen peroxide, organophosphates (e.g., dichlorvos and azamethiphos), ivermectin (and related compounds such as emamectin benzoate), insect molting regulators and pyrethrins (MacKinnon, B. M., World Aquaculture 28: 5-10, 1997; Stone J et al., J Fish Dis 22: 261-270, 1999). Sea lice treatments can be classified into those that are administered by bath (e.g. organophosphates, pyrethrins) and those administered orally (e.g. ivermectin). Bath treatments for sea lice control are difficult, expensive to apply and can have significant effects offish growth following treatments (MacKinnon, supra). Chemicals used in bath treatments are not necessarily effective against all of the stages of sea lice found on fish. At present the use of oral treatments such as emamectin benzoate is predominant in the salmonid industry. Unlike chemicals administered as bath treatments emamectin benzoate does provide short-term protection against re-infection. This treatment although easier to apply than bath treatments is still expensive and, like bath treatments, requires a withdrawal period before animals can be slaughtered for human consumption (Stone, supra). As seen in terrestrial pest and parasites there is evidence for the development of resistance in L. salmonis to some of these treatments, especially in frequently-treated populations (Denholm, I., Pest Manag Sci 58: 528-536, 2002). To reduce the costs associated with sea lice treatments and to eliminate environmental risks associated with these treatments new methods of sea lice control such as vaccines are needed.

A characteristic feature of attachment and feeding sites of caligid copepods on many of their hosts is a lack of a host immune response (Johnson et al., supra; Jones, M. W., et al., J Fish Dis 13: 303-310, 1990; Jónsdóttir, H et al., J Fish Dis 15: 521-527, 1992). This lack of an immune response is similar to that reported for other arthropod parasites such as ticks on terrestrial animals. In those instances suppression of the host immune response is due to the production of immunomodulatory substances by the parasite (Wikel, S. K., et al., “Arthropod modulation of host immune responses”. In The Immunology of Host-Ectoparasitic Arthropod Relationships. Editors: Wikel, S. K., CAB Int., 1996, pp. 107-130). These substances are being investigated for use as vaccine antigens to control these parasites. Sea lice, such as L. salmonis, like other arthropod ectoparasites, produce biologically active substances at the site of attachment and feeding that limits the host immune response. As these substances have potential for use in a vaccine against sea lice we have identified a number of these substances from L. salmonis and have examined their effects of host immune function in vitro.

Potential antigens have been identified using a combination of molecular biological, proteomic, biochemical and immunological techniques. For example, an increase in protease activity has been observed in the mucus of L. salmonis infected Atlantic salmon, compared to non-infected fish (Ross, N. W., et al., Dis Aquat Org 41: 43-51, 2000; Fast, M. D., et al., Dis Aquat Org 52: 57-68, 2002). This increased activity is primarily due to the appearance of a series of low molecular weight (18-24 kDa) proteins that are produced by L. salmonis and were identified as trypsins based on activity, inhibition studies and size. Trypsin activity was identified in infected salmon mucus using aminobenzamidine affinity adsorption and protease zymography (Firth, K. J., et al., J Parasitol 86: 1199-1205, 2000). Several genes encoding for trypsin have been characterized from L. salmonis and the site of trypsin expression determined (Johnson, S. C., et al., Parasitol Res 88: 789-796, 2002; Kvamme, B. O., et al., Int. J. Parasitol. 34, 823-832, 2004; Kvamme, B. O. et al., Gene 352:63-72, 2005).

Several cDNA libraries have been developed from the copepodid, pre-adult and adult stages of L. salmonis. An expressed sequence tag (EST) study of the pre-adult library resulted in the identification of a number of genes encoding trypsin and related proteases (including chymotrypsin and others in the peptidase 51 family), heat shock proteins, cuticle proteins and metabolic enzymes. Some of these genes as described herein have utility as antigens in a sea lice vaccine.

Secretory proteins produced by the sea lice may act as immunomodulatory agents or assist in the feeding activities on the host (Fast, M. D., et al., Exp Parasitol. 107:5-13, 2004; Fast, M. D., et al., J Parasitol 89: 7-13, 2003). Neutralization of these activities by host-derived antibodies may impair sea lice growth and survival on salmon.

Vaccines are generally safer than chemical treatments, both to the fish and to the environment. However, no commercial vaccines against sea lice have been developed to date. Vaccine development has been hindered by a lack of knowledge of the host-pathogen interactions between sea lice and their hosts. There appears to be very limited antibody response in naturally infected hosts. Experimental vaccines, particularly through whole-animal extracts, have been produced against L. salmonis. Investigations in the development of sea lice vaccines have targeted immunogenic proteins from sea lice and, in particular, targeting gut antigens. These vaccines, based on whole animal extracts, have not been shown to be protective though their administration did result in minor changes in L. salmonis fecundity (Grayson T. H., et al., J Fish Biol 47: 85-94, 1995). This particular study, however, was a one-time trial and no further results have been reported from this group.

Accordingly, a first aspect of the present invention provides a protein comprising the amino acid sequence of SEQ ID NO:1 or 2.

Embodiments of the invention provide a recombinant protein comprising the amino acid sequence of SEQ ID NO:1 or 2.

The proteins of the present invention is derived from a secretory/excretory product of L. salmonis.

SEQ ID NO: 1
MAKNKNVGKPRNYKLASGVVRFGKSKMYHKKAIYKFLKKTTPKKVEASK
PAFVEKKVGGAKNGGTRMVRVKKLKNDFPTMERRAHRIAKKPEKLSRRV
RPTLTPGTIAVILAGIHKGKRIVILKELSSGMLLISGPFKLNNCPIRRI
NQRYLLATSTKLDVSSIKMPENINDDYFRRLRAAKKPAGSVFEGKKEEY
KPSEQRKKDQVEVDKQLLNVIMKHPEASLLKQYLKKSFGLSKGQYPHNM
KF
SEQ ID NO: 2
VRFGKSKMYHKKAIYKFLKKTTPKKVEASKPAFVEKKVGGAKNGGTRMV
RVKKLKNDFPTMERRAHRIAKKPEKLSRRVRPTLTPGTIAVILAGIHKG
KRIVILKELSSGMLLISGPFKLNNCPIRRINQRYLLATSTKLDVSSIKM
PENINDDYFRRLRAAKKPAGSV

A second aspect of the invention provides a vaccine against caligid copepod infection in fish, the vaccine comprising an immunologically effective amount of the protein according to the invention or a recombinant protein antigen according to the invention, and a pharmaceutically-acceptable adjuvant, diluent or carrier.

In embodiments of the invention, the vaccine is a recombinant vaccine.

A third aspect of the invention provides the protein, recombinant protein or vaccine according to the invention for use in the treatment or prevention of caligid copepod infection in fish

In embodiments of the invention, the caligid copepod is Lepeophtheirus salmonis.

In embodiments of the invention, the caligid copepod is Caligus rogercresseyi.

In embodiments of the invention, the fish is salmon.

A fourth aspect of the invention provides DNA encoding the amino acid sequence of SEQ ID NO:1 or 2.

In embodiments of the invention, the DNA comprises the nucleotide sequence of SEQ ID NO:3 or 4.

| SEQ ID NO: 3 | |
|---|---|
| 1 | atggcgaaga acaagaacgt cggtaagccg aggaactaca agttagcctc cggagtcgtc |
| 61 | cggttcggca aatctaaaat gtaccacaag aaggcaatct ataaattctt gaagaagaca |
| 121 | actcccaaaa aggttgaggc cagtaagccc gccttcgttg agaagaaggt cggaggtgcc |
| 181 | aagaatgggg gtactcgtat ggttcgcgtc aagaagttga agaacgactt ccccaccatg |
| 241 | gaaagacgtg ctcatagaat cgccaagaag cctgaaaagc tctctcgcag ggtccgtcct |
| 301 | accctcaccc ctggaactat tgcagttatt cttgcaggta tccacaaagg aaagagaatc |
| 361 | gtcattctca aggagctctc cagtggaatg cttctgattt ctggcccctt caagcttaat |
| 421 | aactgcccaa ttagaaggat taatcaacgc tatttgttgg ccacatcaac caagctcgat |
| 481 | gtttcatcca ttaaaatgcc cgagaacatt aatgatgatt acttccgtcg tttaagagcc |
| 541 | gccaagaagc cagctggtag tgtattcgaa ggtaaaaagg aagaatacaa accttctgaa |
| 601 | caacgtaaga aggaccaagt cgaagttgat aagcagctcc tcaatgtcat catgaagcac |
| 661 | cccgaagcct ctcttttgaa acaatacttg aagaagtcct tcggtcttag caagggacaa |
| 721 | tatcctcata atatgaaatt t |
| SEQ ID NO: 4 | |
| 1 | gtccggttcg gcaaatctaa aatgtaccac aagaaggcaa tctataaatt cttgaagaag |
| 61 | acaactccca aaaaggttga ggccagtaag cccgccttcg ttgagaagaa ggtcggaggt |
| 121 | gccaagaatg ggggtactcg tatggttcgc gtcaagaagt tgaagaacga cttccccacc |
| 181 | atggaaagac gtgctcatag aatcgccaag aagcctgaaa agctctctcg cagggtccgt |
| 241 | cctaccctca cccctggaac tattgcagtt attcttgcag gtatccacaa aggaaagaga |
| 301 | atcgtcattc tcaaggagct ctccagtgga atgcttctga tttctggccc cttcaagctt |
| 361 | aataactgcc caattagaag gattaatcaa cgctatttgt tggccacatc aaccaagctc |
| 421 | gatgtttcat ccattaaaat gcccgagaac attaatgatg attacttccg tcgtttaaga |
| 481 | gccgccaaga agccagctgg tagtgta |

A fifth aspect of the invention provides a vaccine against caligid copepod infection in fish, the vaccine comprising an immunologically effective amount of the DNA according to the invention and a pharmaceutically-acceptable adjuvant, diluent or carrier.

A sixth aspect of the invention provides the DNA or the vaccine according to the invention, for use in the treatment or prevention of caligid copepod infection in fish.

In embodiments of the invention, the caligid copepod is Lepeophtheirus salmonis.

In embodiments of the invention, the caligid copepod is Caligus rogercresseyi.

In embodiments of the invention, the fish is salmon.

A seventh aspect of the invention provides an antigen comprising the amino acid sequence of SEQ ID NO:1 or SEQ ID NO:2, or the nucleotide sequence of SEQ ID NO:3 or SEQ ID NO:4.

An eighth aspect of the invention provides the antigen according to the invention for use in the treatment or prevention of caligid copepod infection in fish.

In embodiments of the invention, the caligid copepod is Lepeophtheirus salmonis.

In embodiments of the invention, the caligid copepod is Caligus rogercresseyi.

In embodiments of the invention, the fish is salmon.

A ninth aspect of the invention provides a method of treatment or prevention of caligid copepod infection in fish, comprising administering a therapeutic amount of the protein, recombinant protein, DNA, vaccine or antigen according to the invention, optionally with the co-administration of an adjuvant.

In embodiments of the invention, the caligid copepod is Lepeophtheirus salmonis.

In embodiments of the invention, the caligid copepod is Caligus rogercresseyi.

In embodiments of the invention, the fish is salmon.

As used herein, an “antigen” refers to a molecule containing one or more epitopes that will stimulate a host's immune system to make a humoral and/or cellular antigen-specific response. The term is also used herein interchangeably with “immunogen”.

Adjuvants may be used in vaccines and with antigens of the present invention. Adjuvants which can be used in the context of the present invention include Montanide ISA and IMS Adjuvants (Seppic, Paris, France), other oil-in-water, water-in-oil, and water-in-oil-in-water adjuvants, Ribi's Adjuvants (Ribi ImmunoChem Research, Inc., Hamilton, Mont.), Hunter's TiterMax (CytRx Corp., Norcross, Ga.), aluminium salt adjuvants, nitrocellulose-adsorbed proteins, encapsulated antigens, nanoparticle containing adjuvants. Preferred adjuvants include Seppic Montanide 720, Montanide IMS111x, Montanide IMS131x, Montanide IMS221x, Montanide IMS301x, Montanide ISA206, Montanide ISA 207, Montanide ISA25, Montanide ISA27, Montanide ISA28, Montanide ISA35, Montanide ISA50A, Montanide ISA563, Montanide ISA70, Montanide ISA51, Montanide ISA720, Montanide ISA264. Particularly preferred adjuvants include, Montanide ISA740, Montanide ISA773, Montanide ISA 708, Montanide ISA266. The recommended adjuvant is Montanide ISA763, particularly Montanide ISA 763A VG.

The invention will now be described by way of example with reference to the enclosed Figures, wherein:

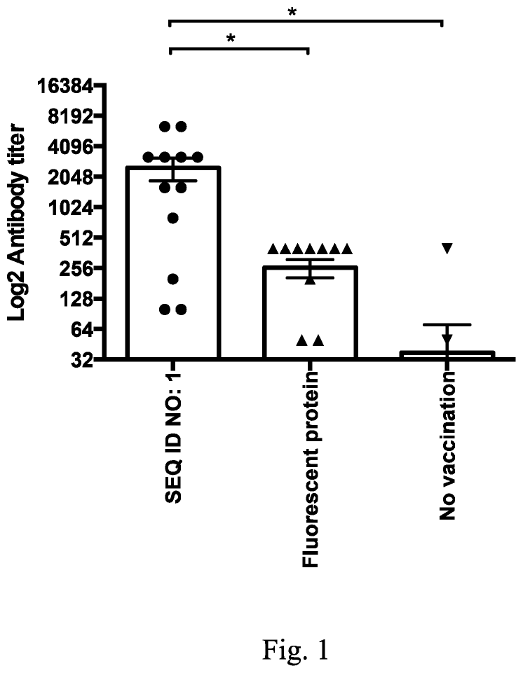

FIG. 1 shows blood serum IgM antibody titer against SEQ ID NO:1 recombinant protein in individual Atlantic salmon parr vaccinated with SEQ ID NO:1 recombinant protein, negative control fluorescent protein or no vaccination control at 602 degree days post-vaccination at 14° C. Individual fish Log₂ titers illustrated with mean±SEM (n=12 fish per group). Data analysed by one-way ANOVA, * signifies significant difference;

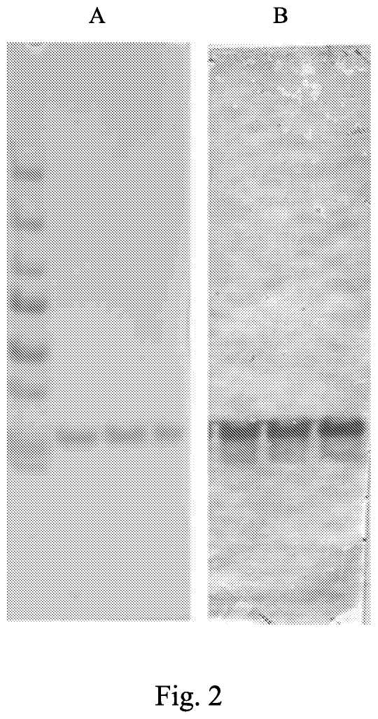

FIG. 2A shows on an SDS-PAGE gel stained with coomassie blue in which one microgram purified SEQ ID NO:4 recombinant protein per well was run;

FIG. 2B shows a western blot of SEQ ID NO:2 recombinant protein probed with serum from Atlantic salmon vaccinated with SEQ ID NO:1 recombinant protein; and

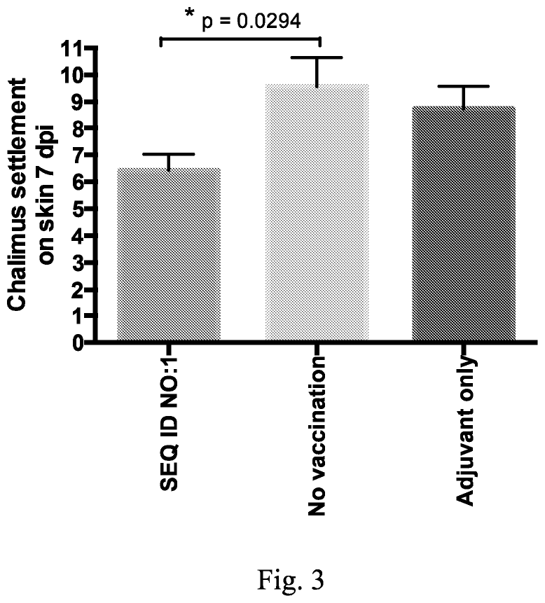

FIG. 3 shows the infection intensity of Lepeophtheirus salmonis chalimus on skin of Atlantic salmon smolts 7 days post-infection by immersion with copepodids at 60 copepodids per fish. Fish were challenged at 1508 degree-days post-prime vaccination with 50 μg recombinant protein SEQ ID NO:1, or adjuvant control containing 250 fluorescent protein, intra-dermally/subcutaneously and 1326 degree-days post-boost vaccination with 50 μg recombinant protein antigen with adjuvant Montanide™ ISA 763 A VG. Vaccination with recombinant protein SEQ ID NO:1 significantly reduced the mean sea lice infection intensity on smolts by 33.3% at 7 days post-infection compared to the no vaccination controls (p=0.0294). Data represented as mean number of chalimus settled on the host's skin±SEM. Data combined from 5 replicate cohabitation tanks, total n per treatment=30.

### EXAMPLES

### Example 1—Maintenance of Atlantic Salmon

Atlantic salmon parr were maintained in a well water flow through system at 13.5±1.0° C. and a dissolved oxygen (DO) level of 9.0±1.0 mg/L. Fish were fed daily at 1.5% of body weight with a commercial dry pellet, and water parameters monitored daily in each tank (temperature, DO, ammonia, nitrite).

After the 602 degree-day post-vaccination sampling day, all fish underwent smoltification by exposure to a 24 hour light regime for 2 weeks followed by the gradual introduction of seawater up to a maximum salinity of 32 ppt. Once the addition of seawater was started the system was switched to a full recirculation system at 13.5±1.0° C. and a dissolved oxygen level of 9.0±1.0 mg/L.

### Example 2—Vaccination of Atlantic Salmon

Atlantic salmon parr were acclimatized in the experimental system for 25 days prior to vaccination. Purified recombinant protein consisting of SEQ ID NO:1 was delivered as a prime intra-dermal vaccination at a dose of 50 μg protein per fish in a total volume of 30 μl in sterile phosphate buffered saline (PBS; 3 injections of 10 μl per fish; n=48 fish; duplicate tanks of 24 fish per group). All fish were tagged under the jaw in order to identify treatment groups. Two hundred and ten degree-days post-prime vaccination, the fish received a booster vaccination containing the corresponding recombinant protein treatment delivered intra-peritoneal at a dose of 50 μg each recombinant protein in a total volume of 100 μl adjuvanted with Montanide ISA 763A VG (Seppic; following manufacturers' instructions). Fish were anaesthetized by immersion in MS-222 at a concentration of 100 mg/L in system seawater buffered with 100 mg/L sodium bicarbonate prior to vaccinations and tagging. Controls included: 1) no vaccination control and 2) control HN-tagged recombinant fluorescent protein at a dose of 250 μg recombinant protein per fish with Montanide ISA 763A VG adjuvant.

At 602 degree-days post-prime vaccination 12 Atlantic salmon were euthanized with 250 mg/L MS-222 buffered with 100 mg/L sodium bicarbonate. Blood sera samples were taken from the caudal vein. The blood was allowed to clot for one hour followed by clot retraction at 4° C. overnight and centrifugation at 3000×g for 7 minutes, and the serum frozen at −80° C. Serum samples were utilized to determine specific antibody titres via ELISA at 602 days post-vaccination. Western blot was performed in order to determine whether or not blood serum from Atlantic salmon immunized with SEQ ID NO:1 recombinant protein cross-reacted to SEQ ID NO:2.

### Elisa

Elisa plates were coated with 100 μl per well 4 μg/ml SEQ ID NO:1 recombinant protein in carbonate:bicarbonate coating buffer (Sigma) overnight at 4° C. Plates were washed three times with low salt wash buffer (LSWB), blocked with 250 μl per well 3% (w/v) casein in PBS overnight at 4° C. Plates were washed again three times with LSWB and 100 μl fish sera diluted in PBS in doubling serial dilutions starting at 1/25 in duplicates were applied to wells, and incubated overnight at 4° C. Plates were subsequently washed five times with high salt wash buffer (HSWB) with a 5 minute incubation on the last wash. One hundred μl primary mouse IgG anti-salmonid IgM antibody (Biorad cat #MCA2182) at 1/500 in PBS was applied to the wells for 1 hour at 22° C., followed by five washes with HSWB and 100 μl of the conjugated secondary goat anti-mouse IgG—HRP (Sigma cat #A4416) at 1/1000 in 1% (w/v) BSA in 1×LSWB for 1 hour at 22° C. The plates were washed again five times with HSWB prior to the addition of 100 μl per well TMB substrate for 10 minutes. The reaction was stopped by the addition of 50 μl 2 M sulphuric acid, and the plates read for absorbance at 450 nm. Each plate contained relevant controls: 1) pooled positive serum, 2) pooled negative plasma, and 3) no serum controls. The titer was calculated as the reciprocal of the dilution that was above the cut-off i.e. above negative control mean A450 nm plus 3×SD. The coefficient of variation of the 450 nm absorbance of sample replicates within a plate, and pooled positive serum between plates was always ≤15%.

### SDS-PAGE

One microgram of SEQ ID NO:2 recombinant protein was loaded onto a Bio-rad mini-protean TGX 4-15% precast gradient gel under reducing conditions with 2-B-mercaptoethanol sample buffer (Bio-rad). Samples were incubated at 95° C. for 5 min in reducing sample buffer. Gels were run at 200 V for 40 minutes followed by staining for 1 hour in 0.25% coomassie blue. Gels were de-stained in 40% methanol, 10% acetic acid until the desired contrast was observed.

### Western Blot

One microgram of SEQ ID NO:2 recombinant protein was loaded onto a Bio-rad mini-protean TGX 4-15% precast gradient gel under reducing conditions with 2-B-mercaptoethanol sample buffer (Bio-rad). Samples were incubated at 95° C. for 5 min in reducing sample buffer. Gels were run at 200 V for 40 min, and then transferred to nitrocellulose membrane (Bio-Rad). Transfers were run at 30 V overnight at room temperature.

Post-blotting, membranes were blocked for 60 minutes in 1% (w/v) BSA in PBS. The membranes were then cut and probed with pooled polyclonal Atlantic salmon serum at 1/100, that was pooled from Atlantic salmon vaccinated with SEQ ID NO:1 recombinant protein, overnight at 4° C. The membranes were subsequently washed 3× with PBS-Tween 20 (0.1%; PBS-T) and then incubated with a monoclonal antibody specific for salmonid IgM at dilutions of 1/500 for 1 hour in 1% BSA in PBS-T. The membranes were subsequently washed three times with PBS-T and incubated with HRP-conjugated goat anti-mouse IgG at 1/3000 in 1% BSA in PBS-T, followed by detection with Opti-4CN™ substrate kit (Bio-rad) following manufacturers' instructions.

### Sea Lice Cultivation

Filtered natural seawater which had undergone reverse osmosis to ensure a salinity of 32 ppt±2 ppt for the sea lice hatchery was obtained from the CCAR facility in Franklin, Me. and added to a 400-litre recirculating system. The system consists of 20 hatching chambers with a fine mesh screen bottom to allow aeration and to hold eggs/larval stages. The hatching chambers were held in 2 litre glass beakers submerged in the system water with an air stone underneath the hatching chamber. Seawater was maintained at a salinity of 32 ppt±2 ppt. The temperature was maintained at 11±1° C. Water quality was monitored and recorded daily for salinity temperature and dissolved oxygen content. The seawater in the beakers underwent a 50% change every other day to ensure good water quality.

Sea lice were collected from a commercial farm harvest. Egg strings from gravid females were removed and placed into the hatching chambers. Egg strings were sorted by coloration to house eggs of similar development together (darker eggs are more developed). Hatching chambers were checked daily to assess larval stages of the sea lice and unhatched egg strings moved to new hatching chambers daily to ensure larvae of similar ages were housed together. To assess larval development, the volume of the water in the hatching chamber was reduced to concentrate the sea lice then duplicate 1 mL samples were taken per hatching chamber. The samples were placed into a petri dish and larval stages were staged and viability assessed using a microscope.

Once the percentage of copepodids reached over 80%, the sea lice were used for infestation. Only copepodids which were 4 days old or less were used for infestations i.e. molted into copepodid ≤4 days ago.

### Sea Lice Infestation

At 602 days post—prime vaccination all Atlantic salmon from the treatment groups were co-housed in six replicate 100 gallon tanks for sea lice challenge (n=6 fish per treatment per tank i.e. 36 fish per treatment total combined). Atlantic salmon were challenged with L. salmonis copepodids at 1508 degree-days post-prime vaccination using a bath challenge method to achieve a final infection level on the no vaccination controls of 10 to 20 lice per fish.

Briefly, the tank volume was lowered to ⅓rd original volume and the copepodids were added to each of the replicate tanks at three different locations to ensure sea lice were distributed throughout the tank to give 60 copepodids per fish. The dissolved oxygen was monitored continuously throughout the 1 hour bath infection to maintain DO at 8.5±0.5 mg/L. After an hour, the water flow was re-instated in order to fill the tank back to its original volume but then left static for a further 1.5 hours prior to re-instating water flow.

### Evaluation of Sea Lice Infection

Seven days post-challenge (once the sea lice reach the late chalimus stage) the Atlantic salmon were euthanized by immersion in 250 mg/L MS-222 in system water until at least 10 minutes post-cessation of opercular movements. The number and stage of sea lice on the skin and gills was counted on each fish using a stereo-microscope. The number of sea lice in each developmental stage was tallied and recorded on the skin and gills separately. The treatment group that the fish belonged to were recorded, i.e. tag colour on left and/or right jaw line, as well as the weight and length of the fish. Skin samples with underlying muscle tissue were taken and fixed in 10% neutral buffered formalin for wax embedding, sectioning and haematoxylin and eosin staining, in order to evaluate the histopathology of the louse attachment site between treatment groups.

### Results

Systemic specific IgM response:

Fish vaccinated with SEQ ID NO:1 recombinant protein were found to possess significantly higher specific IgM antibody titers against SEQ ID NO:1 recombinant protein in the blood serum at 602 degree-days post-vaccination than the fish vaccinated with the fluorescent protein control (p=0.049) or the no vaccination control group (p=0.0257; FIG. 1).
Cross-reaction of serum specific IgM from Atlantic salmon vaccinated with SEQ ID NO:1 recombinant protein with SEQ ID NO:2 recombinant protein:

The blood serum from fish vaccinated with SEQ ID NO:1 recombinant protein contained IgM antibodies at 602 degree-days post-vaccination which recognized SEQ ID NO:2 recombinant protein via Western blot (FIG. 2).
Efficacy:

Vaccination of Atlantic salmon parr with protein SEQ ID NO:1 significantly reduced the mean sea lice infection intensity on the skin of smolts at 7 days post-infection by 33.3% compared to the no vaccination controls (p=0.0294; FIG. 3).

1. A protein comprising the amino acid sequence of SEQ ID NO:1 or 2.

2. A recombinant protein comprising the protein defined in claim 1.

3. A vaccine against caligid copepod infection in fish, the vaccine comprising an immunologically effective amount of the protein according to claim 1, and a pharmaceutically-acceptable adjuvant, diluent or carrier.

4. (canceled)

5. The vaccine according to claim 3, wherein the caligid copepod is Lepeophtheirus salmonis or Caligus rogercresseyi.

6. The vaccine according to claim 3, wherein the fish is salmon.

7. DNA encoding the amino acid sequence of SEQ ID NO:1 or 2.

8. The DNA according to claim 7, wherein the DNA comprises the nucleotide sequence of SEQ ID NO:3 or 4.

9. A vaccine against caligid copepod infection in fish, the vaccine comprising an immunologically effective amount of the DNA according to claim 7 and a pharmaceutically-acceptable adjuvant, diluent or carrier.

10. (canceled)

11. The DNA or vaccine according to claim 9, wherein the caligid copepod is Lepeophtheirus salmonis or Caligus rogercresseyi.

12. The DNA or vaccine according to claim 9, wherein the fish is salmon.

13. An antigen comprising the amino acid sequence of SEQ ID NO:1 or SEQ ID NO:2, or the nucleotide sequence of SEQ ID NO:3 or SEQ ID NO:4.

14. (canceled)

15. (canceled)

16. (canceled)

17. A method of treatment or prevention of caligid copepod infection in fish, comprising administering a therapeutic amount of a protein comprising the amino acid sequence of SEQ ID NO:1 or 2, a recombinant protein comprising the amino acid sequence of SEQ ID NO:1 or 2, a DNA encoding the amino acid sequence of SEQ ID NO:1 or 2, a vaccine against caligid copepod infection in fish, or an antigen comprising the amino acid sequence of SEQ ID NO:1 or SEQ ID NO:2 or the nucleotide sequence of SEQ ID NO:3 or SEQ ID NO:4, optionally with the co-administration of an adjuvant.

18. The method according to claim 17, wherein the caligid copepod is Lepeophtheirus salmonis or Caligus rogercresseyi.

19. The method according to claim 17, wherein the fish is salmon.
